(19) United States
(12) Patent Application Publication
(10) Pub. No.: US 2009/0247751 A1
(43) Pub. Date: Oct. 1, 2009

(54) PROCESS FOR THE PREPARATION OF ZALEPLON AND AN INTERMEDIATE THEREOF

(75) Inventors: Vilas Vasant Pandit, Mumbai (IN); Rajesh Vinodrai Naik, Mumbai (IN); Abhay Atmaram Upare, Mumbai (IN); Rajmohan Shivraj Ubale, Maharashtra (IN)

Correspondence Address: MERCHANT & GOULD PC, P.O. BOX 2903, MINNEAPOLIS, MN, 55402-0903, US

(73) Assignee: PIRAMAL HEALTHCARE LIMITED, MUMBAI (IN)

(21) Appl. No.: 12/280,061

(22) PCT Filed: Feb. 13, 2007
(86) PCT No.: PCT/IB2007/050467

§ 371 Date: Dec. 9, 2008

(30) Foreign Application Priority Data
Feb. 20, 2006 (IN) 241/MUM/2006

(51) Int. Cl. C07D 487/04; C07C 233/43

(52) U.S. Cl. 544/281; 564/220

(57) ABSTRACT

A process for the preparation of N-[3-[3-(dimethylamino)-1-oxo-2-propenyl]phenyl]-N-ethyl acetamide of formula II,

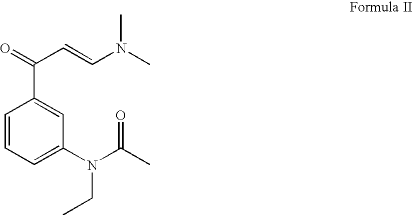

comprising reacting a compound N-[3-[3-(dimethylamino)-1-oxo-2-propenyl]phenyl]acetamide of formula III,

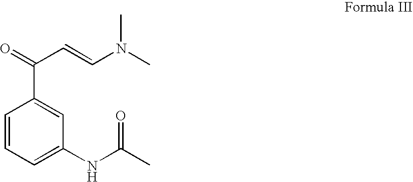

with an ethylating agent in the presence of a strong alkali metal hydroxide and a phase transfer catalyst in a polar-aprotic solvent, and a process for preparation of Zaleplon, which is substantially free of impurities using the said compound of formula II.

### FIELD OF INVENTION

The present invention relates to a process for producing intermediate N-[3-[3-(Dimethylamino)-1-oxo-2-propenyl]phenyl]-N-ethyl acetamide, which is a key intermediate for preparing Zaleplon (N-[3-(3-cyanopyrazolo[1,5-a]pyrimidin-7-yl)phenyl]-N-ethyl acetamide) being a compound of formula I.

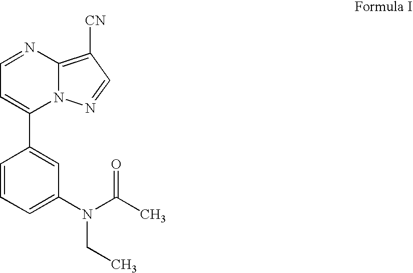

### BACKGROUND OF INVENTION

N-[3-[3-(Dimethylamino)-1-oxo-2-propenyl]phenyl]-N-ethyl acetamide having chemical structure of formula II,

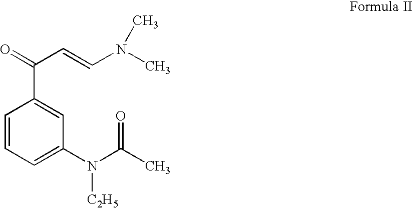

is used in the preparation of Zaleplon. The literature describes many ways of preparing the compound of formula II. U.S. Pat. No. 4,521,422, U.S. Pat. No. 4,654,347 & U.S. Pat. No. 4,626,538 describes alkylation of N-[3-[3-(dimethylamino)-1-oxo-2-propenyl]phenyl]acetamide of formula III, called intermediate I using sodium hydride, ethyl iodide and using dimethylformamide (DMF) solvent. The DMF concentration is a variable used to control the rate of the reaction.

However, sodium hydride is a highly toxic, flammable and corrosive chemical compound, which is an industrial hazard. In contact with water, sodium hydride may explosively liberate hydrogen, and may also produce carbon monoxide and carbon dioxide in the presence of carbon. Therefore, there is a need in the art to provide an industrial process that avoids the use of sodium hydride thereby making the process industrially safer and economical.

WO 03/068775 teaches a process for the preparation of Zaleplon and its intermediates. It teaches N-ethylation of m-acylated acetanilide (3-acetamido acetophenone), by reacting the said starting material with ethyl bromide using pulverized potassium hydroxide (KOH) as the base in tetrahydrofuran (THF). However, the process outlined results in target compound i.e. the intermediate having a purity of only 80-92%, which is not pharmaceutically acceptable.

U.S. Pat. No. 3,886,208 teaches N-alkylation of acylated amines using dialkyl sulfates, using an alkali metal hydroxide as the base and dialkyl sulfoxide as the reaction solvent. However, the process outlined therein affords a yield of only 85-95%.

Therefore, there is a need in the art to provide a process that is not only industrially safe, but is also high yielding with the end products being substantially pure to render them capable of pharmaceutical application.

### OBJECTS OF INVENTION

It is an object of the present invention to prepare the compound of formula II without the use of sodium hydride thereby providing industrially safe process, which is cost effective and can be carried out by using simple and readily available starting materials.

It is a further object of present invention to provide a process capable of producing substantially pure compound of formula II, an intermediate of zaleplon, in good yields.

Another object of the present invention is to provide a process for the preparation of a compound of formula II that produces the said compounds being substantially free of impurities.

### SUMMARY OF THE INVENTION

A process for the preparation of N-[3-[3-(dimethylamino)-1-oxo-2-propenyl]phenyl]-N-ethyl acetamide of formula II,

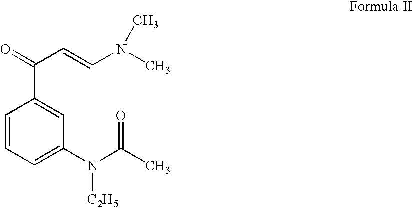

comprising reacting a compound N-[3-[3-(dimethylamino)-1-oxo-2-propenyl]phenyl]acetamide of formula III

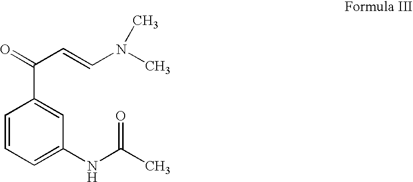

with an ethylating agent in presence of a strong alkali metal hydroxide and a phase transfer catalyst in a polar-aprotic solvent.

In another aspect, the present invention provides a process for the preparation of substantially pure N-[3-[3-(dimethylamino)-1-oxo-2-propenyl]phenyl]-N-ethyl acetamide of formula II,

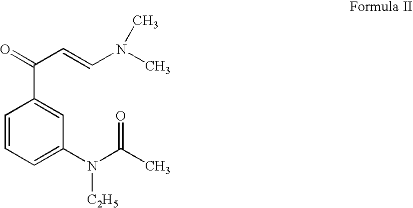

comprising the steps of:
(a) reacting a compound N-[3-[3-(dimethylamino)-1-oxo-2-propenyl]phenyl]acetamide of formula III,

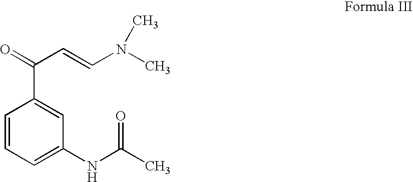

with an ethylating agent in the presence of a strong alkali metal hydroxide and a phase transfer catalyst in a polar-aprotic solvent; and
(b) filtering the reaction mixture obtained in step (a) and thereafter solvent extracting said reaction mixture and washing the product obtained to obtain substantially pure compound of formula II.

In another aspect, the present invention provides a process for the preparation of compound of formula II, said process comprising the steps of:
(a) reacting 3-acetamido acetophenone having formula A,

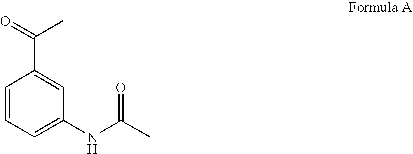

with N,N-dimethylformamide dimethyl acetal having formula B,

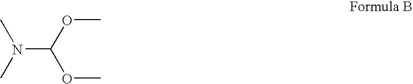

to obtain a compound having formula III;

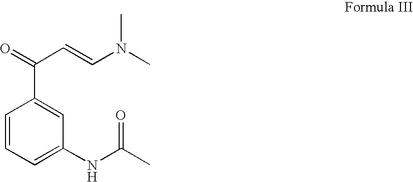

(b) reacting the compound of formula III obtained in step (a) with an ethylating agent in the presence of a strong alkali metal hydroxide and a phase transfer catalyst in a polar-aprotic solvent to obtain a compound of formula II.

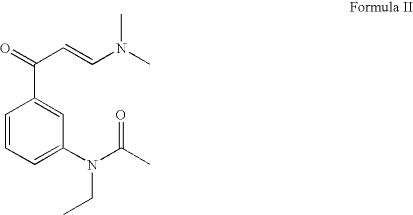

In another aspect, the present invention provides a process for the preparation of Zaleplon having formula I, said process comprising the steps of:
(a) reacting 3-acetamido acetophenone having formula A,

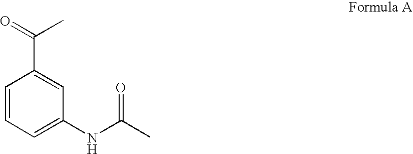

with N,N-dimethylformamide dimethyl acetal having formula B,

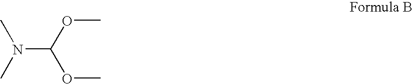

to obtain a compound having formula III,

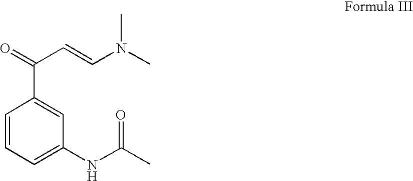

(b) reacting the compound of formula III obtained in step (a) with an ethylating agent in the presence of a strong alkali metal hydroxide and a phase transfer catalyst in a polar-aprotic solvent to obtain a compound of formula II,

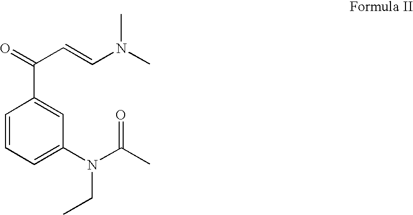

(c) separately reacting malononitrile having formula C,

CH₂(CN)₂ Formula C
with triethylorthoformate having formula D,

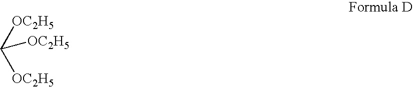

to obtain ethoxymethylene malononitrile having formula E,

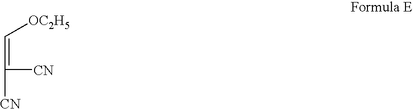

(d) reacting the compound of formula E obtained in step (c) with hydrazine hydrate of formula H₂N—NH₂.H₂O to obtain a compound, 3-aminopyrazole-4-carbonitrile having formula F,

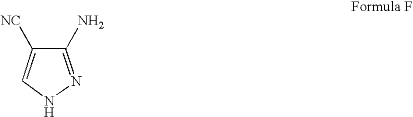

(e) reacting the compound having formula II obtained in step (b) above with the compound of formula F in a mixture of formic acid and acetic acid to obtain Zaleplon represented by formula I,

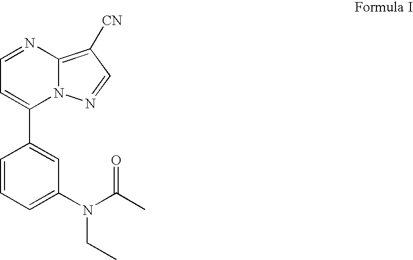

(f) purifying the compound of formula I obtained in step (e) to obtain the substantially pure compound of formula I.

### DETAILED DESCRIPTION OF THE INVENTION

Accordingly, the present invention provides a process for the preparation of N-[3-[3-(dimethylamino)-1-oxo-2-propenyl]phenyl]-N-ethyl acetamide of formula II,

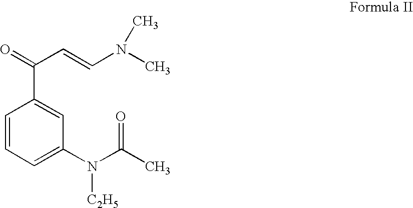

comprising reacting a compound, N-[3-[3-(dimethylamino)-1-oxo-2-propenyl]phenyl]acetamide of formula III,

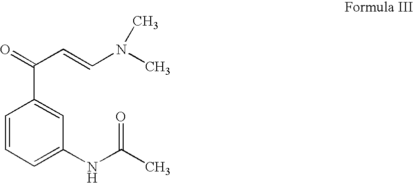

with an ethylating agent in the presence of a strong alkali metal hydroxide and a phase transfer catalyst in a polar-aprotic solvent.

According to a preferred aspect of the invention, the said reaction is carried out in anhydrous conditions.

The said ethylating agent may be a diethyl sulfate or an ethyl halide. In a more preferable embodiment, said ethylating agent may be selected from a group consisting of diethyl sulfate, ethyl bromide, ethyl iodide and ethyl chloride.

The said polar aprotic solvent may be selected from dimethylformamide (DMF) or dimethylsulfoxide (DMSO).

The alkali metal hydroxide may be sodium hydroxide or potassium hydroxide.

The said phase transfer catalyst (PTC) may be selected from a group consisting of tetrabutyl ammonium bromide (TBAB), tetrabutyl ammonium chloride (TBAC) and tetramethyl ammonium bromide (TMAB).

In a preferred embodiment of the invention, the compound of formula III, the ethylating agent and the strong alkali metal hydroxide are used in a molar ratio of 1:1:1 to 1:2:2. The said phase transfer catalyst is used in the molar proportion of between 0.5-2% of the compound of formula III.

The polar aprotic solvent may be preferably used in an amount of between 1-5 times the volume of the compound of formula III.

The reaction may be carried out at temperatures in the range of 5°-40° C. In a preferred aspect, the reaction may be carried out at room temperature.

It has been surprisingly found that the invented process afforded surprising yields and purity levels when the reaction was carried out in the presence of a polar aprotic solvent and a phase transfer catalyst. More surprisingly, only 50% conversion was observed when THF was used as the solvent, and no reaction occurred in THF in the absence of a phase transfer catalyst. Table 1 summarizes the experiments performed with the corresponding yield and purity data.

| | | HPLC/ | | | | | |
|---|---|---|---|---|---|---|---|
| | | TLC | | | | | |
| Sr. | | analysis | Output | Yield | Yield | Purity | |
| No. | Conditions | after 3 hrs. | in gms | Ratio | in % | in % | Remarks |
| 1 | Reaction using | 99.22% | 5.1 | 1.02 | 91 | 99.49 | Reaction was |
| | TBAB (as PTC) | | | | | | completed. |
| | in DMSO solvent | | | | | | |
| 2 | Reaction without | 93.7% | 5.0 | 0.956 | 85.3 | 98.59 | 93% reaction was |
| | TBAB (as PTC) | | | | | | completed in 3 hrs. |
| | in DMSO solvent | | | | | | |
| 3 | Reaction using | 50.62 | 4.0 | 0.8 | 71 | 51.09 | Only 50% |
| | TBAB (as PTC) | | | | | | reaction was |
| | in THF solvent | | | | | | completed |
| 4 | Reaction without | — | 4.3 gm | — | — | — | Reaction did not |
| | using TBAB (as | | | | | | take place. |
| | PTC) in THF | | | | | | |
| | solvent | | | | | | |
| 5 | Reaction using | 95.87% | 4.1 | 0.82 | 73 | 98.87 | Reaction |
| | TBAB (as PTC) | | | | | | completed in 3 |
| | in DMF solvent | | | | | | hours. Rate of |
| | | | | | | | reaction is fast. |
| 6 | Reaction without | 91.3% | 3.2 | 0.64 | 57 | 98 | Reaction |
| | TBAB in DMF | | | | | | incomplete after 3 |
| | solvent | | | | | | hours (on TLC), |
| | | | | | | | completed after 6 |
| | | | | | | | hours after adding |
| | | | | | | | 0.25% excess |
| | | | | | | | ethyl bromide. |

HPLC = High Performance Liquid Chromatography
TLC = Thin Layer Chromatography

The present invention therefore also avoids the use of THF, which is hazardous to use on a large scale. It also avoids the use of sodium hydride, which is moisture sensitive and highly inflammable, thereby hazardous to use on a large scale. The above given table establishes that in the absence of the combination of a polar aprotic solvent and a phase transfer catalyst, the reaction either does not occur or does not reach completion.

In another aspect, the present invention provides a process for the preparation of substantially pure N-[3-[3-(dimethylamino)-1-oxo-2-propenyl]phenyl]-N-ethyl acetamide of formula II,

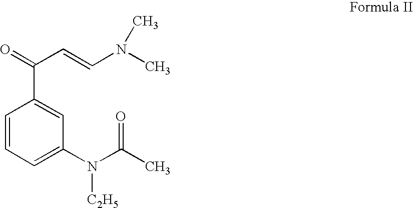

comprising the steps of:
(a) reacting a compound N-[3-[3-(dimethylamino)-1-oxo-2-propenyl]phenyl]acetamide of formula III,

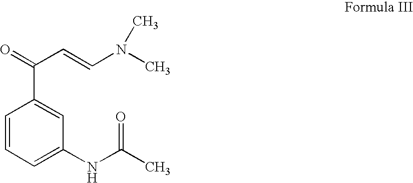

with an ethylating agent in the presence of a strong alkali metal hydroxide and a phase transfer catalyst in a polar-aprotic solvent; and
(b) filtering the reaction mixture obtained in step (a) and thereafter solvent extracting said reaction mixture and washing the product obtained to obtain substantially pure compound of formula II.

The filtration step is additionally performed to avoid the formation of emulsions during the quenching of the reaction system. The target compound of formula II is obtained in substantially pure form by extracting the reaction mixture obtained at the end of step (a) with known organic solvents suitable for such purpose. More preferably, the extraction solvent may be selected from methylene dichloride, chloroform, ethylene dichloride and petroleum ether.

The compound of formula II is used for the preparation of Zaleplon. The entire synthetic route for the preparation of Zaleplon involving the compound of formula II involves six steps designated as step (a) to step (f) respectively. The synthetic scheme is as described hereunder.

In step (a) of the process, the starting material 3-acetamido acetophenone (A) is reacted with N, N-dimethylformamide dimethyl acetal (B) to obtain a compound of formula (III).

The reaction occurring in step (a) may be carried out using processes, which are known in the art.

The compound of formula III obtained in the step (a) above is then reacted according to the present invention as described herein before to provide a compound of formula II in step (b). This reaction according to the present invention is characterized by the combination of a phase transfer catalyst and a polar aprotic solvent in the presence of a strong alkali metal hydroxide base for high yield and purity of the target compound of formula II.

The compound of formula II may be used to prepare the compound Zaleplon having formula I. In step (c), malononitrile having formula CH₂(CN)₂ is separately reacted with triethylorthoformate having formula CH(OC₂H₅)₃ to obtain ethoxymethylene malononitrile having formula E.

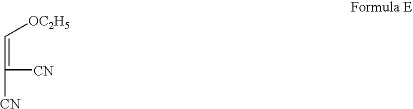

In step (d), the compound of formula E obtained in step (c) is then reacted with hydrazine hydrate (H₂N—NH₂.H₂O) to obtain a compound having the following structural formula F.

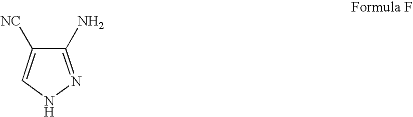

In step (e) of the process, the compound having formula F obtained in step (d) is reacted with the compound of formula II obtained in step (b) in a mixture of formic acid and acetic acid to afford Zaleplon having formula I. In step (f), the compound obtained in step (e) is further purified to afford substantially pure compounds of formula I.

The reactions occurring in steps (c), (d), (e) and (f) may be carried out using conventional processes, which are known in the art.

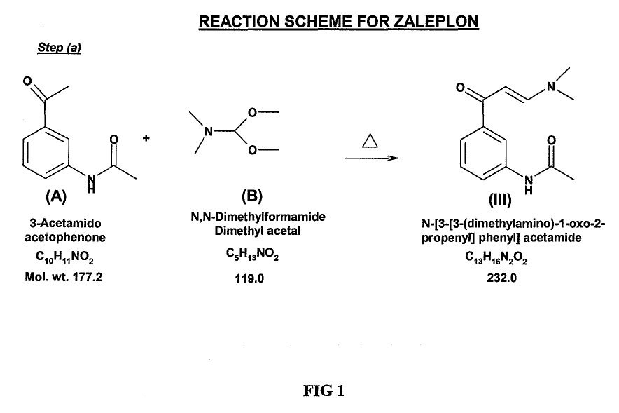

Reference is now drawn to FIG. 1, which illustrates the reaction employed in step (a) of the process for the preparation of Zaleplon and its intermediate, the said compound of formula II.

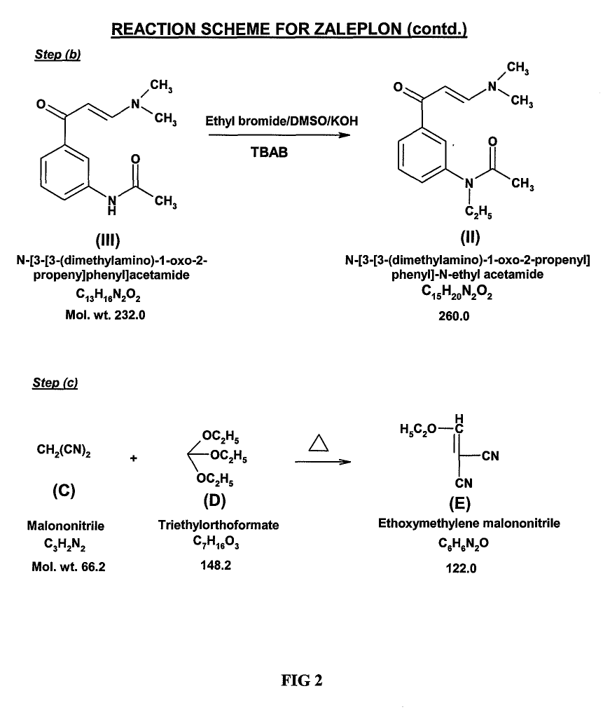

Reference is now drawn to FIG. 2, which illustrates steps (b) and (c) of the said reaction scheme. Step (b) leads to the formation of the said compound of formula II. The step (b) as described herein may optionally include washing the crude intermediate of formula II to obtain substantially pure compound of formula II. The compound obtained in step II is further used to prepare Zaleplon in subsequent steps.

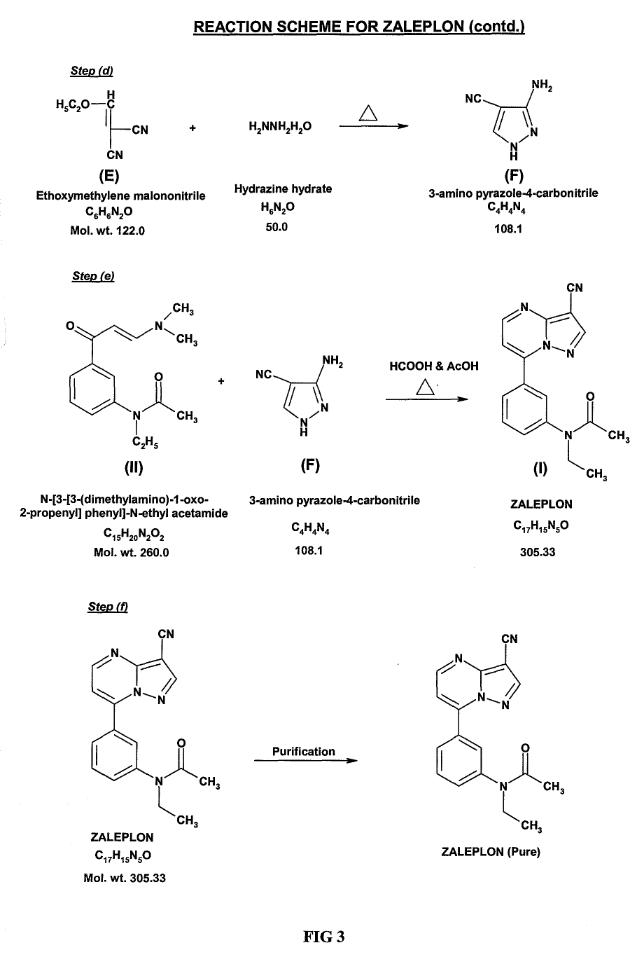

Reference is now drawn to FIG. 3 which illustrates steps (d), (e) and (f) of said reaction scheme. Step (e) involves reacting the compound of formula II obtained in step (b) of the scheme with the compound of formula F obtained in step (d) to obtain Zaleplon having formula I. The crude Zaleplon obtained in step (e) is purified to substantially pure Zaleplon in the step (f) of the scheme.

For obtaining Zaleplon of pharmaceutical grade, it is important that the said reaction in step (b) reaches completion. Any un-reacted compound of formula III, in addition to reducing the overall yield of the process, would react with the compound F in step (e) of the process to produce an impurity, as in the following reaction.

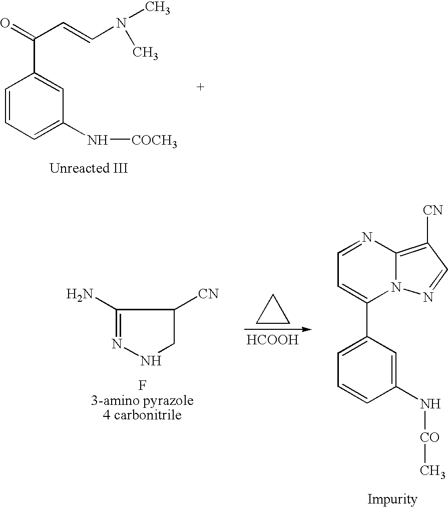

It was surprisingly found that when the said reaction step (b) in the scheme involving reaction of compound of formula III with an ethylating agent was carried out in the presence of alkali metal hydroxide in DMSO and phase transfer catalyst, the crude Zaleplon obtained in step (e) of the scheme was found to be 99.5% pure. The final purified Zaleplon was found to be 99.88% pure with only 0.113% of detectable impurities. Moreover, the only impurity detected in the finally purified compound was the unreacted starting material, which was isolated at step (d) of the process. The following table summarizes the impurity data for the individual process steps, which were likely to result in the formation of impurities.

| Reaction Steps | Purity level | Impurity |
|---|---|---|
| Step (a) | — | — |
| Step (b) | — | — |
| Step (c) | — | — |
| Step (d) | — | 0.001% |
| Step (e) | 99.5% | — |
| Step (f) | 99.88% | 0.113% |

The term “substantially pure” used within the specification is intended to include the referred compounds as being pure to an extent of at least 98%. Thus, substantially pure A should be construed as A having purity greater than or equal to 98%.

The term “ethylation step” used within the appended claims means the step wherein the compound of formula III is reacted with an ethylating agent in the presence of an alkali metal hydroxide in phase transfer catalyst in a polar aprotic solvent.

The invention shall now be explained with reference to the following examples which should not be construed as limiting the scope of the invention. A person skilled in the art appreciates that many modifications for the process may be made without departing from the scope and ambit of the invention.

### Example 1

N-[3-[3-(Dimethylamino)-1-oxo-2-propenyl]phenyl]acetamide (1 kg.) was dissolved in dimethylsulphoxide at 30°±5° C. Potassium hydroxide (powdered) and tetrabutyl ammonium bromide (TBAB), were added and stirred to provide a clear solution. The solution was cooled to 15±3° C. Ethyl bromide was added to the solution while maintaining the temp below 20° C. for 3-4 hrs.

After completion of the reaction, inorganic solid was filtered out. The product was isolated from aqueous layer by extracting with 2-3 times of methylene dichloride. The inorganic solids were washed with methylene dichloride. The combined organic layer was washed with water, dried and charcoalised, filtered and methylene dichloride was removed under vacuum. Hexane was added and mixture of methylene dichloride and hexane was removed under vacuum.

An yellow solid precipitated out and this was cooled to 28°±2° C. and stirred for 2-3 hrs. The solids were filtered out, and washed with n-hexane. The mass was suck dried and then dried at 60° C.

Output: 1 kg. of N-[3-[3-(dimethylamino)-1-oxo-2-propenyl]phenyl]-N-ethyl acetamide.

### Example 2

N-[3-[3-(Dimethylamino)-1-oxo-2-propenyl]phenyl]acetamide (1 kg.) was dissolved in dimethylsulphoxide at 30°±5° C. Potassium hydroxide powder and tetrabutylammonium bromide were mixed and stirred to produce a clear solution. The solution was cooled to 15°±3° C. and diethyl sulphate was added to it at a temperature below 20° C. The mass was stirred at a temperature of 30°±2° C. for 3 hrs. The reaction was monitored on HPLC.

Product isolation was carried out as described in Example 1.

Dry output: 1.04 kg. of N-[3-[3-(dimethylamino)-1-oxo-2-propenyl]phenyl]-N-ethyl acetamide

### Example 3

N-[3-[3-(Dimethylamino)-1-oxo-2-propenyl]phenyl]acetamide (1 kg.) was dissolved in dimethylformamide at 30°±5° C. Potassium hydroxide powder and tetrabutylammonium bromide were mixed to produce a clear solution which was cooled to 15°±3° C. Ethyl bromide was added maintaining the temperature below 20° C. Thereafter the solution was stirred at 30°±2° C. for 3 hrs. TLC showed completion of reaction.

Product isolation was carried out as described in Example 1.

Dry output 1.03 kg. of N-[3-[3-(dimethylamino)-1-oxo-2-propenyl]phenyl]-N-ethyl acetamide.

1. A process for the preparation of N-[3-[3-(dimethylamino)-1-oxo-2-propenyl]phenyl]-N-ethyl acetamide of formula II, 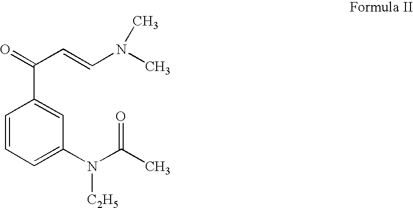
comprising reacting a compound, N-[3-[3-(dimethylamino)-1-oxo-2-propenyl]phenyl]acetamide of formula III, 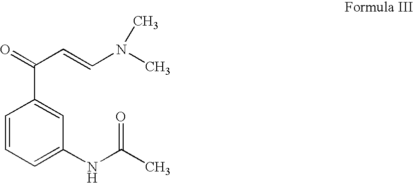
with an ethylating agent in the presence of a strong alkali metal hydroxide and a phase transfer catalyst in a polar-aprotic solvent, with a proviso that the polar aprotic solvent is not tetrahydrofuran.

2. A process for the preparation of compound of formula II, said process comprising the steps of:
(a) reacting 3-acetamido acetophenone having formula A, 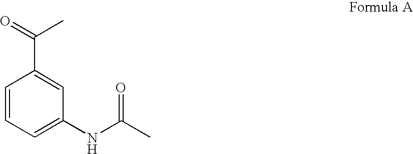
with N,N-dimethylformamide dimethyl acetal having formula B, 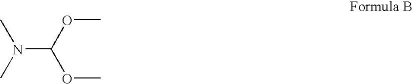
to obtain a compound having formula III; 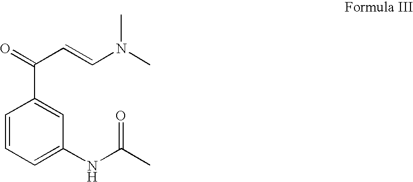
(b) reacting the compound of formula III obtained in step (a) with an ethylating agent in the presence of a strong alkali metal hydroxide and a phase transfer catalyst in a polar-aprotic solvent to obtain a compound of formula II, with a proviso that the polar aprotic solvent is not tetrahydrofuran.

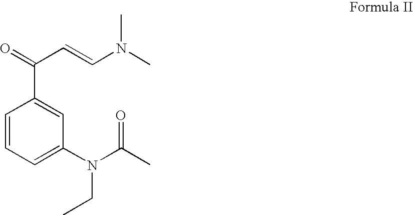

3. A process for the preparation of Zaleplon having formula I, said process comprising the steps of:
(a) reacting 3-acetamido acetophenone having formula A, 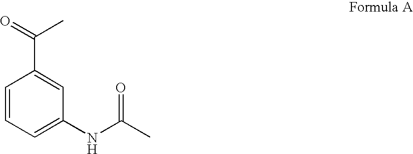
with N,N-dimethylformamide dimethyl acetal having formula B, 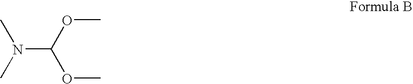
to obtain a compound having formula III, 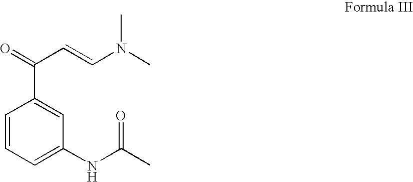
(b) reacting the compound of formula III obtained in step (a) with an ethylating agent in the presence of a strong alkali metal hydroxide and a phase transfer catalyst in a polar-aprotic solvent to obtain a compound of formula II, with a proviso that the polar aprotic solvent is not tetrahydrofuran, 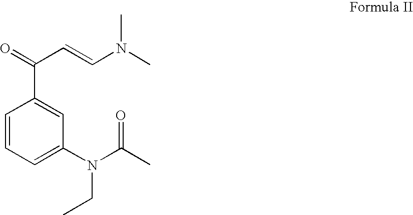
(c) reacting malononitrile having formula C,
CH₂(CN)₂ Formula C
with triethylorthoformate having formula D, 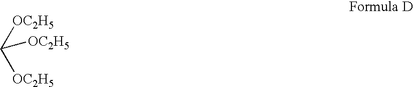
to obtain ethoxymethylene malononitrile having formula E; 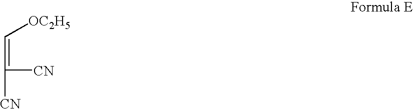
(d) reacting the compound of formula E obtained in step (c) with hydrazine hydrate of formula H₂N—NH₂.H₂O to obtain a compound, 3-aminopyrazole-4-carbonitrile having formula F; 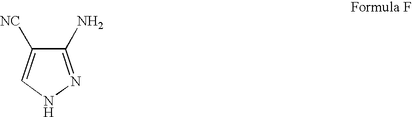
(e) reacting the compound having formula II obtained in step (b) above with the compound of formula F in a mixture of formic acid and acetic acid to obtain a compound of formula I, 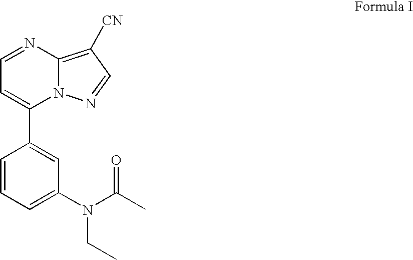
(f) purifying the compound of formula I obtained in step (e) to obtain the substantially pure compound of formula I.

4. A process as claimed in claim 1, wherein the process step of reacting said compound of formula III to obtain said compound of formula II further comprises filtering the reaction mixture obtained at the end of said reaction and thereafter solvent extracting said reaction mixture and washing the product obtained thereby to obtain substantially pure compound of formula II.

5. A process as claimed in claim 4, wherein the solvent for extracting the reaction mixture is selected from a group consisting of methylene dichloride, chloroform, ethylene dichloride and petroleum ether.

6. A process as claimed in claim 1, wherein said step (b) is carried out under anhydrous conditions.

7. A process as claimed in claim 1, wherein said ethylating agent is selected from a diethyl sulfate and an ethyl halide, which is selected from a group consisting of ethyl bromide, ethyl iodide and ethyl chloride.

8. A process as claimed in claim 1, wherein said polar aprotic solvent is selected from dimethyl formamide (DMF) and dimethyl sulfoxide (DMSO).

9. A process as claimed in claim 1, wherein said alkali metal hydroxide is selected from sodium hydroxide and potassium hydroxide.

10. A process as claimed in claim 1, wherein said phase transfer catalyst is selected from a group consisting of tetrabutyl ammonium bromide (TBAB), tetrabutyl ammonium chloride (TBAC) and tetramethyl ammonium bromide (TMAB).

11. A process as claimed in claim 1, wherein said compound of formula III, the said ethylating agent and said alkali metal hydroxide are used in a molar ratio of 1:1:1 to 1:2:2.

12. A process as claimed in claim 1, wherein said phase transfer catalyst is used in a molar proportion of between 0.5-2% of said compound of formula III.

13. A process as claimed in claim 1, wherein said polar aprotic solvent is used in an amount of between 1-5 times the volume of said compound of formula III.

14. A process as claimed in claim 1, wherein the ethylation step is carried out at temperatures between 5°-40° C.

15. A process for the preparation of substantially pure N-[3-[3-(dimethylamino)-1-oxo-2-propenyl]phenyl]-N-ethyl acetamide of formula II, 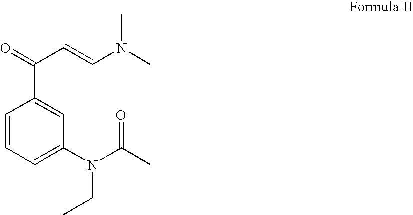
comprising the steps of:
(a) reacting a compound, N-[3-[3-(dimethylamino)-1-oxo-2-propenyl]phenyl]acetamide of formula III, 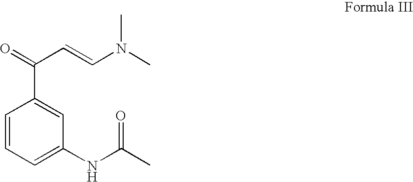
with an ethylating agent in the presence of a strong alkali metal hydroxide and a phase transfer catalyst in a polar-aprotic solvent, with a proviso that the polar aprotic solvent is not tetrahydrofuran; and
(b) filtering the reaction mixture obtained in step (a) and thereafter solvent extracting said reaction mixture and washing the product obtained to obtain substantially pure compound of formula II.

16. A process as claimed in claim 15, wherein said solvent for extracting the reaction mixture is selected from methylene dichloride, chloroform, ethylene dichloride and petroleum ether.

17. A process for the preparation of N-[3-[3-(dimethylamino)-1-oxo-2-propenyl]phenyl]-N-ethyl acetamide of formula II by ethylation of N-[3-[3-(dimethylamino)-1-oxo-2-propenyl]phenyl]acetamide of formula III using ethyl bromide in presence of strong alkali metal hydroxide and tetrabutylammonium bromide in dimethylsulphoxide.

18-19. (canceled)
